(19) United States
(12) Patent Application Publication
(10) Pub. No.: US 2002/0031483 A1
(43) Pub. Date: Mar. 14, 2002

(54) HAIR TREATMENT COMPOSITION COMPRISING A SUGAR OR CARBOHYDRATE FOR THE PURPOSES OF PARTICIPATING IN OXYGEN CONSUMPTION OF A HAIR METHOD AND USE

(76) Inventors: JONATHAN SAMUEL BECK, BEBINGTON (GB); MICHAEL ARTHUR DAVIS, BEDFORD (GB); PREYESH PARMAR, BEDFORD (GB); PAUL SLUSAREWICZ, BEDFORD (GB); GILLIAN ELIZABETH WESTGATE, BEDFORD (GB)

Correspondence Address: UNILEVER, PATENT DEPARTMENT, 45 RIVER ROAD, EDGEWATER, NJ, 07020, US

(21) Appl. No.: 09/400,149

(22) Filed: Sep. 21, 1999

(30) Foreign Application Priority Data
Sep. 22, 1998 (GB) 9820631.1

(51) Int. Cl. A61K 7/06

(52) U.S. Cl. 424/70.1

(57) ABSTRACT

Use in a topical hair composition of a compound selected from a TCA cycle intermediate, an amino acid which is catabolized to be consumed in the TCA cycle, a fatty acid, a glycolysis product, a sugar or a carbohydrate, for the purposes of participating in the oxygen consumption of a hair follicle.

This invention relates to hair treatment compositions containing essential components for hair follicle respiration, and to the use of such components and compositions for continued well being and healthy growth of the hair follicle.

It is known from British patent application number 9704050.5 that cultured hair follicles can synthesise branched and straight chain fatty acids such as 18-MEA and palmitic, stearic and myristic acid if supplied with specific nutrients which are carbon donors for fatty acid chain elongation. These specific nutrients are selected from particular amino acids, sugars, and organic carboxylic acids and their salts, and the associated methods and compositions involve the biosynthesis of hair integral lipid fatty acids in hair follicles. However, this teaching is silent on the mechanisms of respiration in human hair follicles.

We have found that certain groups of compounds can be advantageously applied to the hair in topical compositions, and can participate in the oxygen uptake of the hair follicle. In some circumstances, these compounds can be used to feed and stimulate the central metabolic processes of the follicle, such as respiration of the hair follicle.

Thus, according to a first aspect of the invention, there is provided the use in a topical hair composition of a compound selected from a TCA cycle intermediate, an amino acid which is catabolised to be consumed in the TCA cycle, a fatty acid, a glycolysis product, a sugar or a carbohydrate, for the purposes of participating in the oxygen consumption of a hair follicle.

In certain preferred embodiments, the compound may stimulate the central metabolic processes of the hair follicle, such as respiration of the hair follicle.

In a particularly preferred aspect of the present invention there is provided the use of glucose and/or citrate in a hair composition for the purposes of stimulating respiration of the hair follicle.

Preferably, the hair follicle is a human hair follicle.

After extensive research, we have discovered that the compound referred to are involved in the oxygen consumption process in a hair follicle. Oxygen is important to the cell, since without it the hair follicle has been shown to have retarded growth. Eventually, lack of oxygen to the hair follicle leads to fundamental morphological changes indicative of ejection of the fibre from the follicle. Thus, the provision of one or more of these compounds at active levels in a topical composition for application to the hair provides an ingredient which is involved in oxygen consumption by the hair follicle, and can thereby be said to feed oxygen consumption of the follicle.

The compound for use according to the invention is a TCA cycle intermediate, an amino acid which is catabolised to be consumed in the TCA cycle, a fatty acid, a glycolysis product, a sugar or a carbohydrate, or a mixture thereof.

Suitable TCA cycle intermediates include oxaloacetate, citrate, cis aconitrate, isocitrate, alpha ketoglutarate, succinate, succinyl CoA, fumarate, and malate.

Suitable amino acids which are catabolised to be consumed in the TCA cycle include glutamate, glutamine, histidine, proline, argenine, and other compounds which give rise to alpha ketoglutarate. Additional suitable compounds include isoleucine, methionine and valine, which give rise to succinyl CoA; tyrosine, phenylalanine and aspartate, which give rise to fumarate; asparagine and aspartate, which give rise to oxaloacetate; and alanine, glycine, cysteine, serine, threonine anad tryptophan, which give rise to pyruvate.

Further suitable amino acids include leucine and lysine, which give rise to acetoacetyl CoA, which can be converted to acetyl CoA, and thus enter the TCA cycle.

Suitable fatty acids comprise all fatty acids which can be catabolised by beta oxidation to give rise to acetyl CoA, including those acids which are unsaturated and can be completely oxidised, even if necessary in the presence of the appropriate enzymes (e.g. isomerase, epimerase).

Suitable glycolytic intermediates include fructose 1,6 biphosphate, 1,3 biphosphoglycerate, 2 phosphoglycerate, 3 phosphoglycerate, phosphoenylpyruvate, pyruvate and lactate.

Appropriate sugars include trioses such as glyceraldehyde (aldose) and dihydroxyacetone (ketose), tetroses such as erythrose, threose and erythrulose, and pentoses such as ribose, arabinose, xylose, lyxose, ribulose (plus it's C5 phosphate), and xylolose. Also included are hexoses such as glucose (and it's C6 phosphate), mannose, galactose, fructose (plus it's C1 and C6 phosphates), and sorbose. Also included are the phosphates of the sugars mentioned above, as well as the pyranoses and furanoses into which the pentoses and hexoses can readily be reversibly converted.

Suitable carbohydrates include lactose and sucrose, as well as complex carbohydrates which can be metabolised to provide simple sugars.

Particularly preferred compounds include an aldose, a ketose, glucose, galactose, fructose and citrate, and especially glucose and citrate.

The useful compounds are used at a level which is detectable, effective, and may conveniently be used in topical compositions according to the invention at levels of 0.005-1.0%, more preferably at levels of 0.01-0.5% by weight of the topical composition.

Where the compound is used according to the invention in topical compositions, especially where the topical composition is a shampoo composition, preferably the topical composition comprises at least one surfactant selected from anionic, amphoteric, zwitterionic and cationic surfactants and mixtures thereof.

Advantageously, topical compositions for use according to the invention can be formulated as a shampoo, and will then accordingly comprise one or more cleansing surfactants which are cosmetically acceptable and suitable for topical application to the hair. The invention may also be utilised in conditioner compositions, which are both separate conditioner compositions for topical application, and also so called 2-in 1 compositions containing a shampoo and conditioner.

Suitable cleansing surfactants, which may be used singularly or in combination, are selected from anionic, amphoteric and zwitterionic surfactants, and mixtures thereof.

Examples of anionic surfactants are the alkyl sulphates, alkyl ether sulphates, alkaryl sulphonates, alkanoyl isethionates, alkyl succinates, alkyl sulphosuccinates, N-alkyl sarcosinates, alkyl phosphates, alkyl ether phosphates, alkyl ether carboxylates, and alpha-olefin sulphonates, especially their sodium, magnesium, ammonium and mono-, di- and triethanolamine salts. The alkyl and acyl groups generally contain from 8 to 18 carbon atoms and may be unsaturated. The alkyl ether sulphates, alkyl ether phosphates and alkyl ether carboxylates may contain from 1 to 10 ethylene oxide or propylene oxide units per molecule.

Typical anionic surfactants for use in shampoos of the invention include sodium oleyl succinate, ammonium lauryl sulphosuccinate, ammonium lauryl sulphate, sodium dodecylbenzene sulphonate, triethanolamine dodecylbenzene sulphonate, sodium cocoyl isethionate, sodium lauryl isethionate and sodium N-lauryl sarcosinate. The most preferred anionic surfactants are sodium lauryl sulphate, triethanolamine monolauryl phosphate, sodium lauryl ether sulphate 1 EO, 2EO and 3EO, ammonium lauryl sulphate and ammonium lauryl ether sulphate lEO, 2EO and 3EO.

Examples of amphoteric and zwitterionic surfactants include alkyl amine oxides, alkyl betaines, alkyl amidopropyl betaines, alkyl sulphobetaines (sultaines), alkyl glycinates, alkyl carboxyglycinates, alkyl amphopropionates, alkylamphoglycinates, alkyl amidopropyl hydroxysultaines, acyl taurates and acyl glutamates, wherein the alkyl and acyl groups have from 8 to 19 carbon atoms. Typical amphoteric and zwitterionic surfactants for use in shampoos of the invention include lauryl amine oxide, cocodimethyl sulphopropyl betaine and preferably lauryl betaine, cocamidopropyl betaine and sodium cocamphopropionate.

The cleansing surfactant(s) may be present in shampoo compositions of the invention in a total amount of from about 1 to about 40% by weight based on the total weight of the shampoo composition, preferably from about 2 to about 30% by weight, optimally from about 10% to 30% by weight.

The shampoo can also include nonionic surfactants to help impart aesthetic, physical or cleansing properties to the composition. The nonionic surfactant can be included in an amount ranging from 0% to about 5% by weight based on total weight.

For example, representative nonionic surfactants that can be included in shampoos of the invention include condensation products of aliphatic (C₈-C₁₈) primary or secondary linear or branched chain alcohols or phenols with alkylene oxides, usually ethylene oxide and generally having from 6 to 30 ethylene oxide groups.

Other representative nonionics include mono- or di-alkyl alkanolamides. Examples include coco mono- or di-ethanolamide and coco mono-isopropanolamide.

Further nonionic surfactants which can be included in shampoos for the invention are the alkyl polyglycosides (APGs). Typically, the APG is one which comprises an alkyl group connected (optionally via a bridging group) to a block of one or more glycosyl groups. Preferred APGs are defined by the following formula:

RO—(G)ₙ

wherein R is a branched or straight chain alkyl group which may be saturated or unsaturated and G is a saccharide group.

R may represent a mean alkyl chain length of from about C₅ to about C₂₀. Preferably R represents a mean alkyl chain length of from about C₈ to about C₁₂. Most preferably the value of R lies between about 9.5 and about 10.5. G may be selected from C₅ or C₆ monosaccharide residues, and is preferably a glucoside. G may be selected from the group comprising glucose, xylose, lactose, fructose, mannose and derivatives thereof. Preferably G is glucose.

The degree of polymerisation, n, may have a value of from about 1 to about 10 or more. Preferably, the value of n lies in the range of from about 1.1 to about 2. Most preferably the value of n lies in the range of from about 1.3 to about 1.5.

Suitable alkyl polyglycosides for use in the invention are commercially available and include for example those materials identified as: Oramix NS10 ex Seppic; Plantaren 1200 and Plantaren 2000 ex Henkel.

Likewise the composition can include other emulsifiers, conditioning agents, inorganic salts, humectants and similar materials to provide the composition with desirable aesthetic or physical properties.

Representative conditioning agents that can be included in shampoos of the invention include silicones. Typically these are present in the composition at a level of from 0.01% to 10%, preferably from 0.5 to 5%, by weight based on total weight.

Silicones are particularly preferred conditioning agents for hair. Representative silicones include volatile and non-volatile silicones, such as for example polyalkylsiloxanes (optionally end-capped with one or more hydroxyl groups), polyalkylaryl siloxanes, siloxane gums and resins, cyclomethicones, aminofunctional silicones, quaternary silicones and mixtures thereof.

Preferred silicones include polydimethylsiloxanes (of CTFA designation dimethicone) and hydroxylated polydimethylsiloxanes (of CTFA designation dimethiconol). Suitably the average particle size of the silicone in the shampoo composition is less than 20 microns and preferably less than 2 microns. Particle size may be measured by means of a light scattering technique, using a 2600D Particle Sizer from Malvern Instruments. The silicone is preferably emulsion-polymerised, since this enables silicones of very high viscosity to be more easily processed. The silicone can be cross-linked.

Silicones of the above types are widely available commercially, for example as DC-1784 and DCX2-1391, both ex Dow Corning.

Shampoo compositions of the invention may also include a polymeric cationic conditioning compound that is substantive to the hair and imparts conditioning properties to the hair.

The polymeric cationic conditioning compound will generally be present at levels of from 0.01 to 5%, preferably from about 0.05 to 1%, more preferably from about 0.08% to about 0.5% by weight. Synthetic or naturally derived polymers having a quaternised nitrogen atom are useful. The molecular weight of the polymer will generally be between 5 000 and 10 000 000, typically at least 10 000 and preferably in the range 100 000 to about 2 000 000.

Representative synthetic quaternised polymers include, for example: cationic copolymers of 1-vinyl-2-pyrrolidine and 1-vinyl-3-methyl-imidazolium salt (e.g., Chloride salt) (referred to in the industry by the Cosmetic, Toiletry, and Fragrance Association, “CTFA”. as Polyquaternium-16); copolymers of 1-vinyl-2-pyrrolidine and dimethylaminoethyl methacrylate (referred to in the industry by CTFA as Polyquaternium-11); cationic diallyl quaternary ammonium-containing polymers including, for example, dimethyldiallylammonium chloride homopolymer (referred to in the industry (CTFA) as Polyquaternium 6); mineral acid salts of amino-alkyl esters of homo-and co-polymers of unsaturated carboxylic acids having from 3 to 5 carbon atoms, as described in U.S. Pat. No. 4,009,256; and cationic polyacrylamides as described in WO095/22311.

Representative naturally-derived quaternised polymers include quaternised cellulosic compounds and cationic guar gum derivatives, such as guar hydroxypropyltrimonium chloride. Examples are JAGUAR C-13S, JAGUAR C-15, and JAGUAR-C17, commercially available from Meyhall in their JAGUAR (trademark) series.

A shampoo composition for use according to the invention can also include optional conditioning agents such as branched chain fatty acids, such as 18-MEA, or straight chain fatty acids such as palmitic, myristic and/or stearic acids. In general these ingredients can be included in an amount ranging from 0% to about 3% by weight based on total weight.

Topical compositions for use in accordance with the invention may also be formulated as a hair conditioner for the treatment of hair (typically after shampooing) and subsequent rinsing. Such formulations will then accordingly comprise one or more conditioning surfactants which are cosmetically acceptable and suitable for topical application to the hair, and which compositions may also be used according to the invention.

Suitable conditioning surfactants are selected from cationic surfactants, used singly or in admixture. Examples include quaternary ammonium hydroxides or salts thereof, e.g. chlorides.

Suitable cationic surfactants for use in hair conditioners of the invention include cetyltrimethylammonium chloride, behenyltrimethylammonium chloride, cetylpyridinium chloride, tetramethylammonium chloride, tetraethylammonium chloride, octyltrimethylammonium chloride, dodecyltrimethylammonium chloride, hexadecyltrimethylammonium chloride, octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, and the corresponding hydroxides thereof. Further suitable cationic surfactants include those materials having the CTFA designations Quaternium-5, Quaternium-31 and Quaternium-18. Mixtures of any of the foregoing materials may also be suitable. A particularly useful cationic surfactant for use in hair conditioners of the invention is cetyltrimethylammonium chloride, available commercially, for example as GENAMIN CTAC, ex Hoechst Celanese.

In hair conditioners of the invention, the level of cationic surfactant is preferably from 0.01 to 10%, more preferably 0.05 to 5%, most preferably 0.1 to 2% by weight of the composition.

Conditioners of the invention advantageously incorporate a fatty alcohol material. The combined use of fatty alcohol materials and cationic surfactants in conditioning compositions is believed to be especially advantageous, because this leads to the formation of a lamellar phase, in which the cationic surfactant is dispersed.

Representative fatty alcohols comprise from 8 to 22 carbon atoms, more preferably 16 to 20. Examples of suitable fatty alcohols include cetyl alcohol, stearyl alcohol and mixtures thereof.

The level of fatty alcohol materials is conveniently from 0.01 to 10%, preferably from 0.1 to 5% by weight of the composition. The weight ratio of cationic surfactant to fatty alcohol is suitably from 10:1 to 1:10, preferably from 4:1 to 1:8, optimally from 1:1 to 1:4.

Conditioners for use according to the invention can include other emulsifiers, conditioning agents, inorganic salts, humectants and similar materials to provide the composition with desirable aesthetic or physical properties. Silicones, as described above for shampoo compositions, are particularly preferred conditioning agents for hair.

As further optional components for inclusion in shampoo or conditioner compositions for use according to the invention, in addition to water, may be mentioned the following conventional adjunct materials known for use in cosmetic compositions: suspending agents, thickeners, pearlescing agents, opacifiers, salts, perfumes, buffering agents, colouring agents, emollients, moisturisers, foam stabilisers, sunscreen materials, antimicrobial agents, preservatives, antioxidants, natural oils and extracts, propellants.

The compositions of the invention are primarily intended for topical application to the hair and/or scalp of a human subject to participate in oxygen consumption by the hair follicle.

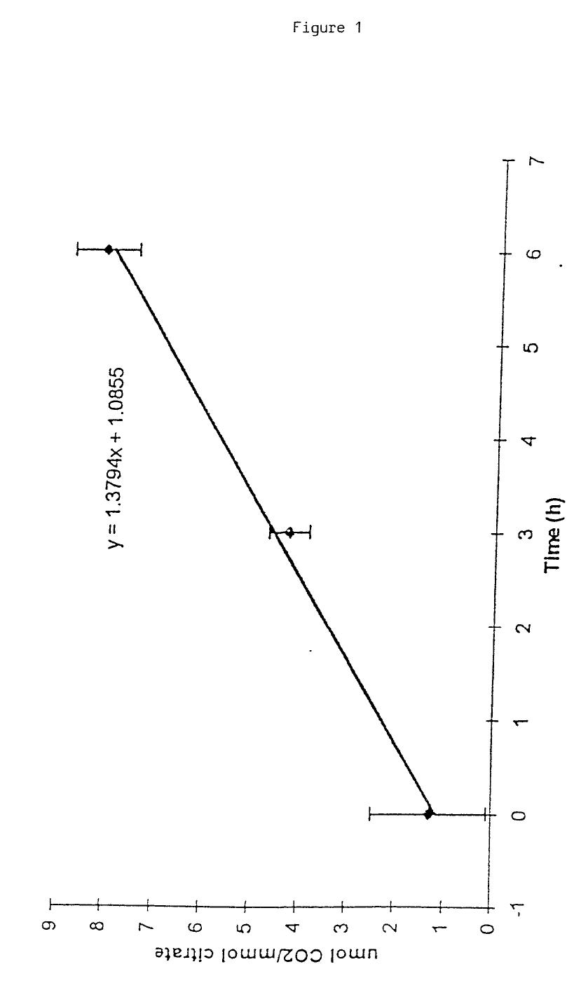

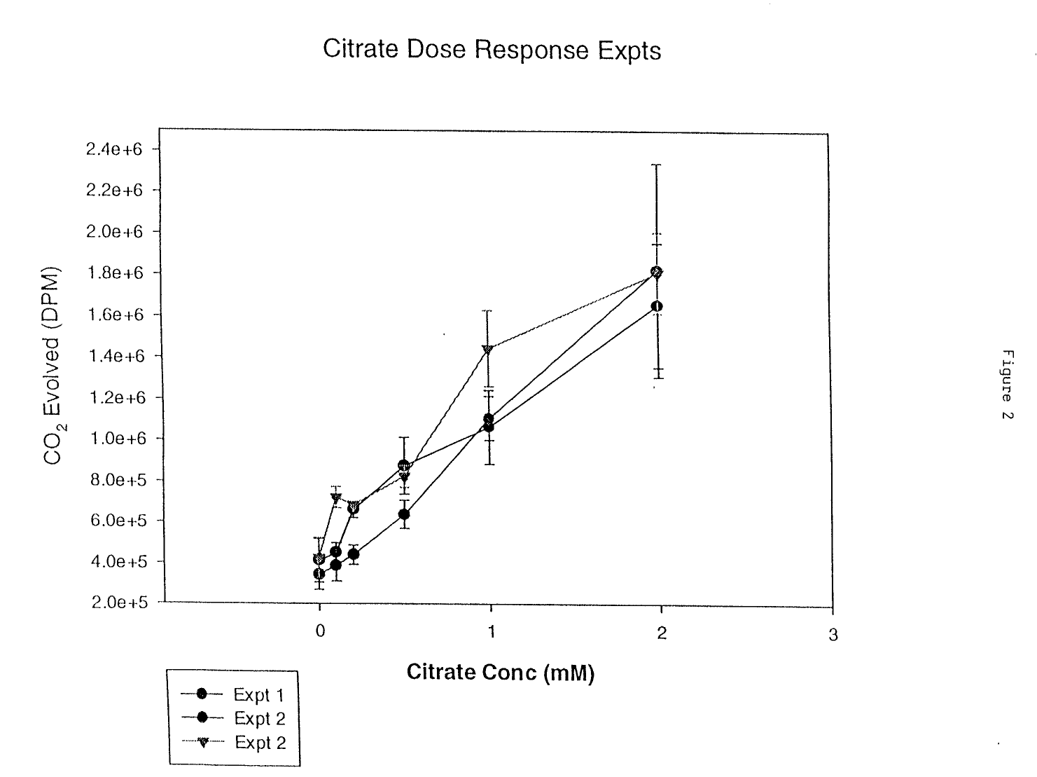

The invention will now be further illustrated by the following non-limiting Examples, FIG. 1, which shows a graph of the amount of radiolabelled CO₂ emitted with time, from Example 3, and FIG. 2, which shows graphically the results of the citrate dose response experiment.

### EXAMPLE 1

Example 1 demonstrates how hair follicles utilise a medium which contains the described compounds in order to consume oxygen from the atmosphere.

Follicles were isolated from human facelift skin and grown in culture according to the methods described (Philpott et al J.Cell.Sci.97: pp 463-470 1990). For the oxygen consumption experiments a Clarke electrode apparatus (Tank Brothers Limited, Cambridge, England) was used.

The equipment was set up in accordance with the manufacturers instructions, the temperature of the cell being maintained at 37° C. using a circulating water bath. The output of the electrode was recorded using Perkin Elmer Turbochrom 4 data logging software.

The apparatus contains a Teflon membrane in the cell which was changed daily and the cell calibrated and prepared for use as follows. 2 ml of the medium to be used for the experiment (a derivative of William's E culture medium containing 1.5 mM HEPES), thoroughly gassed using 5% carbon dioxide in air, was added to the stirred cell. Following recording of the initial baseline the output from the Rank Brothers electronics was set to 1000 mV. Oxygen was then purged by bubbling pure nitrogen through the medium in the cell. After recording this new ‘zero oxygen’ baseline the medium was reoxygenated by bubbling air through and the new baseline recorded. This procedure provided a check that the electrode was working correctly, and gave a value for zero oxygen which was used in subsequent calculations of oxygen consumption. A value of 6.7 mg/lt for the oxygen concentration in air equilibrated water (medium) at 37° C. was used in all calculations. (This value was taken from a table of values supplied with a commercial oxygen metering apparatus). No additional corrections were made for salinity or for altitude.

After calibration, a fresh 2 ml sample of William's E medium gassed with 5% carbon dioxide in air was added, the plunger inserted into the cell ensuring that no air bubble was trapped, and the output from the cell recorded to give a rate for oxygen consumption of the cell. When this baseline had been established the plunger was removed from the cell, the follicles added and the plunger reinserted. The new rate of oxygen consumption was recorded for a sufficient time as to enable its accurate determination. Inhibitors were injected into the measurement cell using a long needle fitted to a microsyringe. 50 μl of sodium azide solution at 2.6 mg/ml in medium was injected into 2 ml medium in the cell to give a final concentration of 2 mM. Alternatively, Strophanthidin was used as a 2 mM solution in DMSO, injecting 20 μl to give a final concentration of 0.1 mM.

From the calibration of the electrode, a curve was derived from which a baseline reading corresponding to zero oxygen level could be derived.

In the experiment, 160 follicles were added to 2 ml of medium. On addition of the follicles to the medium, a rapid increase in oxygen consumption was observed. However, a rapid reduction of oxygen consumption was also noticed on addition of an inhibitor to the medium. Rates of oxygen consumption by follicles ranged between 6 and 82 pmol/minute/follicle, which is in line with theoretical estimates based on glucose and glutamine uptake from Williams E medium. In the various repetitions of the example, inhibition of oxygen consumption by the follicles by 2 mM sodium azide was typically 54-87% of the oxygen consumption, whilst for 0.1 mM strophanthidin it was 76% inhibition of the oxygen consumption.

### EXAMPLE 2

In a further experiment demonstrating the essential nature of oxygen, parallel sets of hair follicles are incubated for up to 6 days, in the presence or absence of oxygen, and the growth of the follicles observed.

In this example follicles from three individuals were isolated.

Twenty-four hair follicles from each sample were placed into 24-well sterile culture plates (Nunclon; one follicle per well). One plate of follicles from each skin sample was then incubated at 37° C. under an atmosphere of 2.5%CO₂/97.5% air. Duplicate plates were also incubated in the same incubator, but these had been sealed in a modular incubator chamber (Flow Laboratories). The chamber contained moistened tissue to maintain humidity and the air within it had been replaced by 2.5%CO₂/97.5% nitrogen.

Follicular length was measured under an inverted microscope with a calibrated eyepiece graticule immediately after isolation (day 0) and after 1, 2, 4 and 6 days in culture.

Follicles which did not grow at all during culture were excluded from the experiment. After each measurement the atmosphere in the chamber was renewed with fresh 2.5%CO₂/97.5% nitrogen or 2.5%CO₂/97.5% air.

Follicles incubated in the presence of oxygen grew in a linear manner, which was not the case for those incubated anaerobically. Those incubated anoxically grew at approximately half the rate of those grown in an oxygen containing atmosphere. Additionally, those growth anoxically showed no signs of growth after four days, with the onset of necrosis visible as early as two days into the experiment. Necrotic regions were clearly visible in the follicular bulbs four days into the experiment, together with abnormal bulb morphology.

### EXAMPLE 3

This example demonstrates the involvement of suitable compounds such as glucose and citric acid in the oxygen consumption of the follicle.

Hair follicles were isolated from human scalp skin obtained from four different female subjects; hair follicles were isolated from human scalp skin and placed into a 6 well culture plate at a density of 15 follicles/well in 5 mL complete Williams E medium containing penicillin (50 μg/mL), streptomycin (50 IU/mL), L-glutamine (2 mM), hydrocortisone (10 ng/mL) and insulin (10 μg/mL). In addition a cocktail of amino acids consisting of arginine (0.45 mM) proline (1 mM), glycine (2.6 mM) and alanine (1 mM) was also added since these are though to improve the maintenance of hair follicles in vitro. After an overnight incubation (37° C., 5% CO₂ in air) to ensure continued growth following the isolation procedure, follicles were cut to a uniform length and incubated until required.

For each experimental time point, five follicles were transferred to each of 3 eppendorf tubes in triplicate with 10 μL medium. Control samples containing 100 μL medium and no hair follicles were also assayed in triplicate for each time point. At the beginning of the experimental period 100 μL of medium, as above, containing approximately 3 μCi [1,5-¹⁴C]-sodium citrate (120 mCi/mmol, Amersham International plc, Buckinghamshire) was added to each eppendorf. The tubes were then placed in glass scintillation vials (Beckman) and sealed with rubber suba seal stoppers (15 mm Philip Harris code S-76-516). The scintillation vials were gassed with 5% CO₂ in air and incubated at 37° C. After the appropriate incubation period 1 mL of KOH (30% w/v) was injected into the vials outside the eppendorft tube. Perchloric acid (100 μL) was then injected into the eppendorf in order to displace CO₂ dissolved in the medium. The vials were then shaken gently overnight to allow uptake of CO₂ into the KOH. After the seals had been removed the medium was sampled for counting the remaining radioactivity (3×10 μL). The KOH solution was analysed by liquid scintillation counting (Beckman LS 60001C) after addition of 18 mL Hionic Fluor (Packard).

Results from the mean of each triplicate control were subtracted from the corresponding mean from the experimental results. These combined results were then analysed by regression. In order to confirm that individual time points were significantly different from one another a one way analysis of variance was carried out and pairwise comparison conducted by using the Student-Newman-Keuls method utilising Sigmasta™ software.

During a six hour period there was continuous evolution of radiolabelled CO₂ and an excellent correlation was obtained when the results are expressed as radiolabel dpm liberated (R²=0.9991).

Initial (time 0 h) results were obtained by placing follicles into eppendorf tubes as for all other time points, sealing and gassing them before addition of KOH and perchloric acid. Control (0 h) results were then subtracted from the follicle results. Consequently some negative results were obtained when controls were slightly higher than follicle results, illustrating the experimental variability inherent in the methodology. There were marginal differences in the amount of radiolabelled citrate added in the different experiments. As a result a similar but more accurate reflection of metabolism can be gained when CO₂ evolution is expressed as a function of citrate concentration (FIG. 1). There is a slight amount of volatile citrate present in the radiolabelled medium as shown by the immediate apparent evolution of CO₂ at the start of the experiment (time 0 h).

Metabolism of citrate led to a linear increase in the amount of CO₂ evolved during the experimental period with an average production of 0.23 (±0.04) μmol CO₂ up to 12 h (FIG. 3), the maximum length of time looked at, due to the requirement for high specific activity of the added [1,5-¹⁴C] citrate and therefore the limited amount of medium/follicle that could be used. The results are shown graphically in FIG. 1.

Experiments were also conducted to establish the metabolism rate of citrate, using a similar protocol to that above, except that all incubations were carried out for 6 hours, and the samples differed from each other by containing a different amount of unlabelled citrate (i.e. 0.0, 0.1, 0.2, 0.5, 1.0 and 5.0 mM). The results of this are shown graphically in FIG. 2.

The results indicate that citrate is taken up into hair follicles and used as an energy source.

Qualitatively similar results were obtained when radiolabelled glucose was incorporated in the eppendorf tubes in place of sodium citrate.

### EXAMPLES 4-9

Examples 4-9 represent suitable topical compositions according to the invention:

| CTFA Name | 4 | 5 | 6 | 7 | 8 | 9 |
|---|---|---|---|---|---|---|
| Sodium Laureth Sulfate | 14.16 | 20.48 | 20.48 | 20.00 | 20.00 | 20.00 |
| Cocamidopropyl Betaine | 5.33 | 5.33 | 5.33 | 5.33 | 5.33 | 5.33 |
| Pearlizer | 12.50 | 10.00 | 10.00 | | 0.20 | 0.20 |
| Dimethicone Conditioner | 2.00 | 1.60 | 0.80 | 0.80 | 6.00 | 4.00 |
| Guar Hydroxypropyl | 0.20 | 0.10 | 0.10 | | 0.10 | 0.10 |
| Trimonium Chloride | | | | | | |
| Polyquaternium 10 | | | | 0.10 | | |
| Carbomer 940 | 0.20 | | | | 0.40 | 0.40 |
| Preservative | 0.19 | 0.19 | 0.19 | 0.20 | 0.20 | 0.20 |
| L-Isoleucine | 0.01 | 0.01 | 0.01 | 0.01 | 0.01 | 0.01 |
| D-Glucose | 0.10 | 0.10 | 0.10 | 0.10 | 0.10 | 0.10 |
| Sunscreen | | | | 0.10 | | |
| Perfume | 0.50 | 0.50 | 0.50 | 0.50 | 0.50 | 0.50 |
| Antioxidant | 0.05 | 0.05 | 0.05 | 0.05 | 0.05 | 0.05 |
| Sodium Hydroxide | 0.04 | | 0.02 | | 0.24 | 0.24 |
| Sodium Chloride | 1.00 | 0.30 | 0.30 | 0.85 | 0.20 | 0.20 |
| Citric Acid | 0.20 | 0.20 | 0.20 | 0.20 | 0.20 | 0.20 |
| Dye | | | | trace | | |
| Water | to | to | to | to | to | to |
| | 100% | 100% | 100% | 100% | 100% | 100% |

1. Use in a topical hair composition of a compound selected from a TCA cycle intermediate, an amino acid which is catabolised to be consumed in the TCA cycle, a fatty acid, a glycolysis product, a sugar or a carbohydrate, for the purposes of participating in the oxygen consumption of a hair follicle.

2. Use in a topical hair composition of a compound selected from a TCA cycle intermediate, an amino acid which is catabolised to be consumed in the TCA cycle, a fatty acid, a glycolysis product, a sugar or a carbohydrate, for the purposes of stimulating respiration of a hair follicle.

3. Use according to claim 1 or 2, wherein the compound is an aldose, a ketose, glucose, galactose, fructose and/or citrate.

4. Use according to any of the preceding claims, wherein compound is used at a level of 0.005-1.0% by weight of the topical hair composition.
